(19) United States
(12) Patent Application Publication
(10) Pub. No.: US 2017/0172667 A1
(43) Pub. Date: Jun. 22, 2017

(54) CANNULA WITH OPTICAL SENSING

(71) Applicant: NOVARTIS AG, Basel (CH)

(72) Inventor: STEVEN T. CHARLES, MEMPHIS, TN (US)

(21) Appl. No.: 15/051,095

(22) Filed: Feb. 23, 2016

Related U.S. Application Data
(60) Provisional application No. 62/268,347, filed on Dec. 16, 2015.

(51) Int. Cl. A61B 34/20; A61B 17/02

(52) U.S. Cl. CPC A61B 34/20; A61B 17/0231; A61B 2034/2057; A61B 2034/2065

(57) ABSTRACT

An apparatus for detecting a type of ophthalmic tool at a surgical site includes a cannula having an elongated body arranged to be introduced into an eye. The body may include a lumen extending therethrough. The lumen may be arranged to allow a shaft of an ophthalmic tool to fit therethrough. The apparatus may also include an optical waveguide having an end facing the lumen and an optical transceiver assembly in optical communication with the optical waveguide. The optical transceiver assembly may include an optical sensor and a light source configured to direct light transmitted through the optical waveguide and into the lumen.

### TECHNICAL FIELD

The present disclosure is directed to methods and systems that are applicable to ophthalmology. More particularly, the present disclosure is directed to methods and systems involving use of cannulas in ophthalmic medical procedures.

### BACKGROUND

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibrils that are often attached to the retina. Therefore, cutting and removal of the vitreous must be done with great care to avoid traction on the retina, the separation of the retina from the choroid, a retinal tear, or, in the worst case, cutting and removal of portions of the retina itself. Delicate operations such as cutting and removal of vitreous near a mobile, detached portion of the retina, vitreous base dissection, and cutting and removal of membranes are particularly difficult.

The use of microsurgical cutting probes in posterior segment ophthalmic surgery is known. Such vitrectomy probes are typically inserted through a cannula and into the posterior segment. The cannula is typically a hollow tube having a central lumen through which ophthalmic tools may be introduced into the eye. The cannula itself is inserted into an eye through use of a trocar. The trocar fits within the central lumen of the cannula and includes a needle that extends from the distal end of the cannula and is used to puncture the eye. The cannula is introduced with the trocar and slides into the opening created by the needle. Removing the trocar leaves the cannula in place, providing an access port through tissue.

An operator may then insert a variety of ophthalmic tools through the cannula and into the eye. Such tools may include a fiber optic illuminator, an infusion cannula, an aspiration probe, or a vitrectomy probe. Some of these may be plugged into and powered or controlled by a surgical console. A user interface on the surgical console may allow the user to operate the ophthalmic tools that are plugged into the surgical console. For example, through an input mechanism such as a foot pedal, an operator may cause a vitrectomy tool that is plugged into the console to cut and aspirate vitreous tissue.

In some cases, multiple tools may be simultaneously connected to a surgical console, and a user must separately switch between the connected tools in order to designate one of the tools as the active tool. Once designated as the active tool, the tool can be operated. When a user wishes to utilize a different tool connected to the surgical console, the user must manually select the different tool as the active tool in order to operate the different tool.

### SUMMARY

According to one example, an apparatus for detecting a type of ophthalmic tool at a surgical site includes a cannula having an elongated body arranged to be introduced into an eye, the body comprising a lumen therethrough, the lumen arranged to allow a shaft of an ophthalmic tool to fit therethrough. The apparatus also includes an optical waveguide having an endpoint facing the lumen and an optical transceiver assembly in optical communication with the optical waveguide. The optical transceiver assembly includes a light source configured to direct light through the optical waveguide and into the lumen and an optical sensor.

According to one example, a method for identifying which ophthalmic tool of a plurality of ophthalmic tools is inserted into a cannula includes inserting a cannula into an eye, the cannula comprising an optical waveguide having an endpoint at an interior of the cannula. The method further includes inserting an ophthalmic tool into the cannula, the ophthalmic tool comprising a shaft having a marking. The method further includes sensing light from within the interior of the cannula through the optical waveguide. The method further includes identifying the ophthalmic tool based on a pattern in the light reflected from the tool shaft, the pattern being produced as the marking passes the endpoint.

According to one example, a system for detecting a type of ophthalmic tool at a surgical site includes a console having a control system and a plurality of ophthalmic tool ports. The system further includes a plurality of ophthalmic tools arranged to connect to the ports, each ophthalmic tool comprising a shaft having a unique marking. The system further includes a cannula having an optical waveguide with an endpoint directed at an interior of the cannula. The system further includes an optical transceiver assembly in optical communication with the optical waveguide. The optical transceiver assembly includes an optical sensor in communication with the control system, a light source adapted to direct light into the optical waveguide, and a beam splitter to direct light from the optical waveguide to the optical sensor.

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is a diagram showing an illustrative ophthalmic surgical system.

FIG. 2A is a schematic diagram of an illustrative apparatus that includes a cannula with optical sensing.

FIG. 2B is a schematic diagram of the apparatus shown in FIG. 2A in which the cannula is within an eye and an ophthalmic tool is within the cannula.

FIG. 3 is a schematic diagram showing an illustrative surgical console that utilizes an optical sensing cannula.

FIGS. 4A and 4B show cross-sectional views of illustrative cannulas with multiple optical waveguides disposed within.

FIG. 5A is a diagram showing a cross-sectional view of an optical sensing cannula that is partially inserted into an eye.

FIG. 5B is a diagram showing a cross-sectional view of an optical sensing cannula that is fully inserted into an eye.

FIG. 6 is an example flowchart showing an illustrative method for identifying a tool that is inserted into an optical sensing cannula.

FIG. 7 is an example flowchart showing an illustrative method for using an optical sensing cannula to determine whether the cannula is appropriately positioned.

FIG. 8 is a cross-sectional view of a cannula having waveguides having ends disposed in a common transverse plane.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

As described above, ophthalmic surgical procedures often involve the use of a variety of ophthalmic tools. One or more of the ophthalmic tools may be connected to and/or powered by a surgical console. For example, one or more of the ophthalmic tools may be plugged into and powered or controlled by a surgical console. In some instances, one or more of the ophthalmic tools may be wirelessly connected to a surgical console, and the surgical console may control one or more functions of the ophthalmic device via wireless communication. A user interface on the surgical console may allow the user to operate the ophthalmic tools that are plugged into the surgical console. For example, through an input mechanism such as a foot pedal, an operator may cause a vitrectomy tool that is connected into the console to cut and aspirate vitreous tissue.

To more efficiently and safely manage such tools, principles described herein relate to methods and systems for determining whether a tool is currently within the eye. If a tool is determined to be in the eye, methods and systems described herein may involve identifying that tool and establishing the identified tool as the active tool. This may allow the user to switch the active tool controlled by the console without requiring manual inputs at the console. According to one example of principles described herein, the cannula through which ophthalmic tools enter the eye includes a sensor that may determine which instrument has been inserted through or is disposed within the cannula. In one implementation, the cannula includes an optical waveguide, such as an optical fiber, having an end within the central lumen of the cannula. The optical waveguide may be in optical communication with an optical transceiver assembly that directs a light source into the optical waveguide. The optical transceiver assembly also may include an optical sensor to sense light passing back through the optical waveguide. In addition, a shaft of an ophthalmic tool connected to the surgical console may include a marking. The marking may represent an optical machine-readable representation of data such as a laser marking or barcode that represents a unique pattern. When the marking passes by the end of the optical waveguide, the marking is reflects back light. The optical sensor within the optical transceiver assembly may detect the marking based on the reflected light and may identify the tool in the cannula. Although in some instances the marking provided on the ophthalmic tool may be a laser marking or barcode, the scope of the disclosure is not so limited. Rather, the marking provided on the instrument may be any desired or suitable marking. For example, the marking may be a type of data matrix code, magnetic ink character code, or any other suitable code.

The ability to determine that an ophthalmic tool is within an eye, as well as the ability to identify that tool, may provide a variety of benefits to a user and a patient. For example, in some instances, it may be desirable that certain operations of certain tools not be used while the tool is within the eye. For example, when a vitrectomy tool is within the eye, it is important not to use the vitrectomy tool's back-flush operation because doing so may traumatize or cause damage or infection in the eye, or create other health problems. If the surgical console recognizes that the vitrectomy tool is within the eye, then the surgical console may be configured to automatically disable the back-flush functionality. Additionally, the ability to identify the tool may allow the surgical console to automatically configure itself to control whichever tool is within the eye. For example, the foot pedal may be set to operate the particular tool identified by the optical sensing cannula. This may also speed the surgical process resulting in shorter surgeries, which may cause patient health and recovery advantages.

A cannula with such a sensor may provide other benefits as well. For example, the level of light detected by the optical sensor may be used to determine whether the cannula is properly inserted in the eye. For example, if the distal tip of the cannula is within the suprachoroidal space, then only a small amount of light may be reflected back through the optical waveguide. In contrast, when the distal tip of the cannula is fully within the eye, a relatively larger amount of light will be reflected back through the optical waveguide. The optical sensor may be able to detect such differences in light levels. Based on detected light levels, the surgical console may be operable to determine whether the cannula is appropriately positioned within an eye. In some instances, the surgical console may notify a user if the cannula is not properly inserted. Additionally, the surgical console may disallow use of any tool within the cannula if the cannula is not properly inserted.

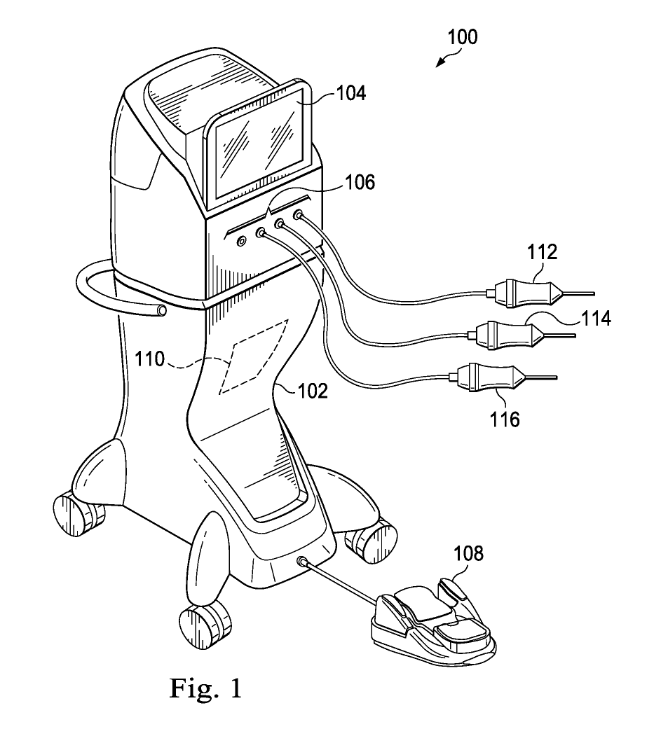

FIG. 1 is a diagram showing an illustrative ophthalmic surgical system 100. According to the present example, the ophthalmic surgical system 100 includes a surgical console 102. The surgical console 102 may include a display screen 104, a plurality of ophthalmic tool ports 106, and an input device 108. In this example, the input device 108 is a foot pedal. However, other input devices may also be used. For example, input devices such as switches, buttons, triggers, touchscreen elements, keyboards, mice, and others may also be used. The ophthalmic surgical system 100 further includes a plurality of ophthalmic tools 112, 114, and 116. Any or all of the ophthalmic tools 112, 114, and 116 may be connected to the console 102. For example, one or more of the ophthalmic tools 112, 114, and 116 may be plugged into the ophthalmic tool ports 106. In some implementations, the surgical console 102 is designed to be mobile and may be used by a user, such as a health care provider, to perform ophthalmic surgical procedures. The surgical console 102 may also include a control system 110 that may be configured to process, receive, and store data and provide signals to one or more of the ophthalmic tools 112, 114, and 116 and/or the display screen 104.

The display screen 104 may communicate information to the user, and in some implementations, may show data relating to system operation and performance during a surgical procedure. In some implementations, the display screen 104 may display data related to a specific one of the ophthalmic tools connected to the surgical console 102. For example, the display screen 104 may display data related to the active tool. In some examples, the display screen 104 is a touchscreen that allows the operator to interact with the surgical console 102 through a graphical user interface.

The ophthalmic tool ports 106 are adapted to allow a variety of ophthalmic tools to be plugged thereinto. The ophthalmic tool ports 106 may include a variety of connection types. For example, the ophthalmic tool ports 106 may include fluid source connections to provide fluids to an ophthalmic tool and thus to the eye, pneumatic connections to supply power to pneumatically driven tools, and electrical connections to both power and communicate electronically with an ophthalmic tool.

In some examples, the input device 108 may be used to operate only one of the ophthalmic tools connected to the surgical console 102 at a time. In one example, a user may designate one of the ophthalmic tools 112, 114, or 116 as the tool to be controlled by the input mechanism. In some cases, when there are multiple active tools, the user may associate one of the active tools with the input device 108. Once assigned, the input device 108 is operable to control the designated active tool. The user may also use a separate input mechanism (not shown) to control a different active tool. In some implementations, the user may assign an active tool for control by the input device 108 through the graphical user interface associated with the display screen 104.

As mentioned above, an ophthalmic surgical procedure may involve the use of a plurality of different surgical tools including, for example, the vitrectomy probe 112, the infusion tool 114, and the imaging tool 116. A variety of other types of ophthalmic tools such as a vitreous cutter, endoilluminator, aspiration cannula, fragmenter, endolaser, diathermy device, scissors, forceps, and infusion cannula may be used as well. The surgical console 102 may be configured to detect which ophthalmic tool is connected to an ophthalmic tool port 106. For example, in one implementation, the plug on one or more of the ophthalmic tools 112, 114, and 116 that connects to the surgical console 102 may include a Radio Frequency Identifier (RFID) tag. The surgical console 102 may include an RFID reader that may read the RFID code produced by the RFID tag and identify the ophthalmic tool that has been plugged into a specific ophthalmic tool port 106. Thus, in some implementations, the operator does not have to manually input the identity or type of the ophthalmic tool into the surgical console 102.

The ability to determine what tools are plugged into a surgical console does not determine whether those tools are within a patient's eye and in use. A tool connected to the surgical console 102 may not be inserted into an eye. For example, a plugged-in tool may be outside the eye being primed, tested, back-flushed by an operator, or otherwise connected to the surgical console 102 but not inserted into the eye. In a further example, a tool may be connected to the surgical console 102 but may be lying on a drape. Through use of principles described herein, the surgical console 102 may determine if one of the tools that are plugged into the surgical console 102 is within the eye.

In some examples, the user may provide input to the surgical console 102 that indicates to the surgical console 102 an eye (for example, the left eye or the right eye) into which a cannula is placed. The user may also provide input to the surgical console 102 that indicates to the surgical console 102 the position of the cannula (e.g., at a nasal or a temporal location within the eye). In some cases, multiple cannulas embodying principles described herein may be in use simultaneously.

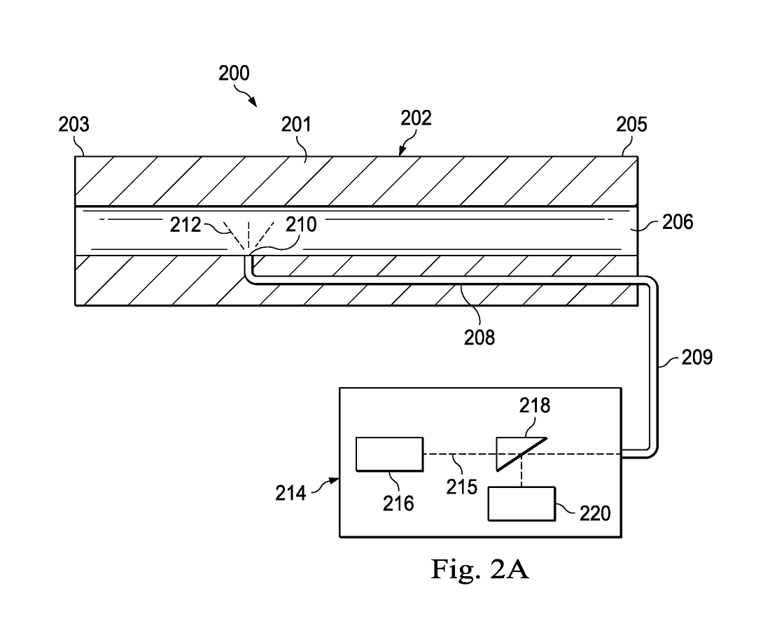

FIG. 2A is an illustrative schematic diagram of a tool identifying apparatus 200 that may be used to identify the type of ophthalmic tool being used during a surgical procedure. The tool identifying apparatus 200 includes a cannula 202 having optical sensing and an optical transceiver assembly 214. The cannula 202 is in optical communication with the optical transceiver assembly 214 through an optical cable 209.

The cannula 202 includes an elongated hollow body 201 with a distal end 203 and a proximal end 205. The cannula 202 includes a central lumen 206 arranged to receive a trocar and/or the shaft of an ophthalmic tool. The body 201 of the cannula 202 includes an optical waveguide 208. The optical waveguide 208 may be embedded within the body 201. The optical waveguide 208 is optically coupled to the optical cable 209. In some implementations, the optical waveguide 208 is an optical fiber. For example, in some instances, nanofibers may be used. Other types of optical waveguides may also be used. In some implementations, the optical cable 209 and the optical waveguide 208 may be a single, unitary component. For example, in some instances, the optical cable 209 and the optical waveguide 208 may be or include a continuous optical fiber. In other implementations, the optical cable 209 and the optical waveguide 208 may be separate components that are optically coupled such that light traveling through one is transmitted to and carried by the other.

The optical waveguide 208 is operable to transmit light from the optical transceiver assembly 214 into the central lumen 206 as well as transmit light received from the within the central lumen 206 to the optical transceiver assembly 214. The optical waveguide 208 includes a first end 210. The first end 210 of the optical waveguide 208 defines a surface, and the surface of the first end 210 may form a portion of an inner wall of the cannula that defines the central lumen 206. Thus, the first end 210 terminates at the central lumen 206. As such, the optical waveguide 208 is in optical communication with the central lumen 206 via the first end 210. Light 212 transmitted from the optical transceiver assembly 214 and through the optical waveguide 208 is directed into the central lumen 206 of the cannula 202 via the first end 210. The first end 210 also receives light from within the central lumen 206. As explained above and in further detail below, the light from within the central cannula 206 that is received by the first end 210 of the optical waveguide 208 may be reflected from a surface of an instrument received within the central lumen 206 of the cannula 202.

The optical transceiver assembly 214 includes a light source 216, a beam splitter 218, and an optical sensor 220. The light source 216 produces light 215 that is directed into the optical cable 209 and thus into the optical waveguide 208. In some implementations, the light source 216 may be a laser. In some implementations, the light source 216 may be a Light Emitting Diode (LED). However, the scope of the disclosure is not so limited. Rather, any suitable light source may be used.

Some of the light 215 produced by the light source 216, and projected into the central lumen 206 of the cannula 202, may be reflected back into the optical waveguide 208. For example, a portion of the light 215 may be reflected off of an instrument present within the lumen 206 and into the first end 210 of the optical waveguide 208 and carried therethrough. The reflected light is carried along the optical cable 209 and received by the optical transceiver assembly 214. The beam splitter 218 is used to redirect at least a portion of the reflected light to the optical sensor 220. In some implementations, the optical sensor 220 may be a photodiode. A photodiode produces an electric current in response to impinging light. The strength of the electric current may be proportional to the strength of the impinging light. The optical sensor 220 may be in communication with a control system, such as the control system 110 of the surgical console 102 shown in FIG. 1.

In some implementations, the optical transceiver assembly 214 may be integrated with the surgical console 102. In other implementations, the optical transceiver assembly 214 may be a discrete component separate from the surgical console 102. In such implementations, the optical transceiver assembly 214 may be in communication with the surgical console 102 so that data from the optical sensor 220 may be provided to the surgical console 102. Such communication may be wired or wireless.

FIG. 2B is an illustrative schematic diagram of the tool identifying apparatus 200 in which the cannula 202 is disposed within an eye 226 and an ophthalmic tool 228 is present within the central lumen 206 of the cannula 202. As described above, the cannula 202 may be inserted into an eye 226 through use of a trocar (not shown). After the trocar is removed, any variety of types of ophthalmic tools may be inserted into the eye 226 through the central lumen 206 of the cannula 202. As shown in FIG. 2B, the ophthalmic tool 228 includes a shaft 222 that is arranged to fit within the central lumen 206 of the cannula 202. The shaft 222 of the ophthalmic tool 228 may be inserted into the proximal end 205 of the cannula 202 and advanced until a distal end 229 of the shaft 222 extends past the distal end 203 of the cannula 202. The ophthalmic tool 228 may then be used to perform its intended operation within the eye 226. The ophthalmic tool 228 may correspond to any of the ophthalmic tools 112, 114, and 116 described above, for example.

According to the present example, the shaft 222 includes a marking 224 that is used by the tool identifying apparatus 200 to identify the ophthalmic tool 228. The marking 224 may be unique to a type of ophthalmic tool inserted into the cannula 202. In some instances, for example, the marking 224 may be unique to a vitrectomy probe. In some cases, the marking 224 may be unique to a specific model of vitrectomy probe. The marking 224 may be formed in any manner that may allow it to be identified by the tool identifying apparatus 200. In some implementations, the marking 224 may be made of a material that has a different reflectivity than all or a portion of the remainder of the shaft 222. In some instances, the marking 224 may be more reflective than the rest of the shaft 222. In other instances, the marking 224 may be less reflective than all or a portion of the rest of the shaft 222. In some implementations, differences in reflectivity between the marking 224 and all or a portion of the remainder of the shaft 222 may be the result of differences in color, differences in surface roughening or etching, or other physical characteristics. As the marking 224 passes by the endpoint 210 of the optical waveguide 208, the light reflected back through the optical waveguide 208 is affected. Specifically, the pattern of the marking 224 causes a corresponding variation in the light signal detected by the optical sensor 220. For example, the variation in light may form a pattern recognized by the control system, such as control system 110 for example. In some cases, the marking 224 may be an engraving formed in the cannula 202. The engraving may reflect light differently and thus affect the light reflected back through the optical waveguide 208.

The variation in reflected light detected by the optical sensor 220 may be used to identify the ophthalmic tool 228 received into the cannula 202. As described above, the optical sensor 220 may communicate with the control system 110 of the surgical console 102. For example, the control system 110 may compare a detected light pattern with a database of light patterns. The database may associate particular light patterns with particular ophthalmic tools. By matching the detected light pattern to an entry within the database, the corresponding ophthalmic tool may be determined.

In some examples, as the ophthalmic tool 128 is removed from the eye, the marking 224 passes by the first end 210 of the optical waveguide 208. Thus, upon removal of the ophthalmic tool 228, a variation in reflected light, such as a pattern, caused by the marking 224 is transmitted to the optical transceiver assembly 214. The optical transceiver assembly 214 can then send a signal to a control system, such as the control system 110 of the surgical console 102, for example. Consequently, the reflected light may be used to determine that the ophthalmic tool 228 has been removed from the eye 226.

In some implementations, more than one tool identifying apparatus 200 may be used at a given time. For example, two separate cannulas may be simultaneously disposed within the eye 226, and a control system, such as control system 110, is operable to identify a tool inserted into each of the cannulas.

In other implementations, information such a particular eye of a patient on which a surgical procedure is to be perform (e.g., the right or left eye), a position of a user (e.g., a surgeon) relative to the patient, and locations of the eye into which each of the cannulas is to be or has been inserted may be input into a surgical console, such as the example surgical console 102. Based on this information, along with the detection of an ophthalmic tool being inserted or removed from the cannula as described herein may enable the surgical console to detect which of the user's hands (e.g., the right hand or left hand) is currently holding a tool.

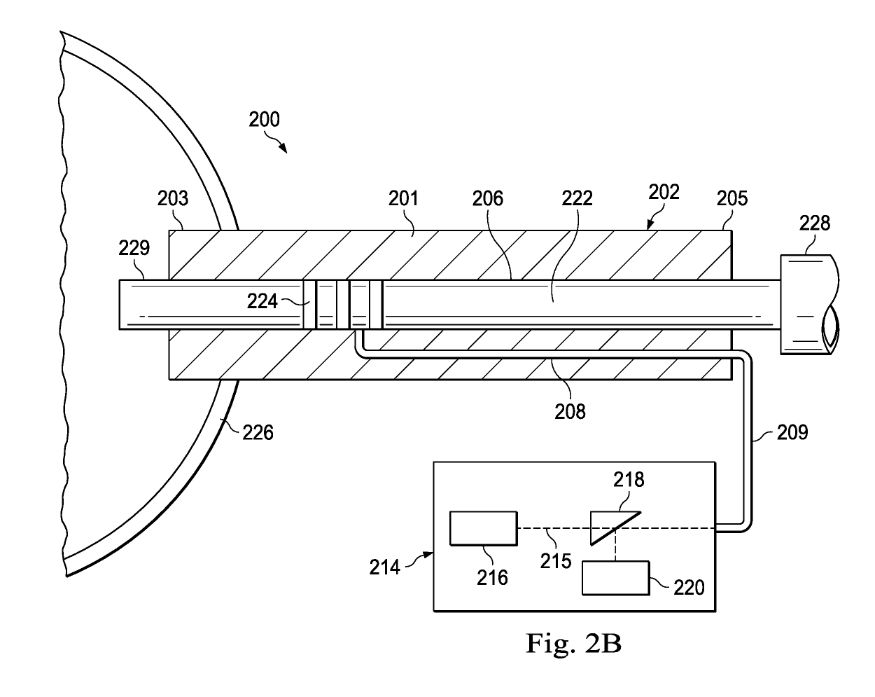

FIG. 3 is a schematic diagram showing the surgical console 102 that includes the control system 110. The surgical console 102 is coupled to tool identifying apparatus 200. As explained above, the tool identifying apparatus 200 may include the cannula 202 and the optical transceiver assembly 214. The surgical console 102 is communicatively coupled to the optical transceiver assembly 214 and is operable to identify the type of surgical tool from a plurality of surgical tools based on reflected light received from the tool identifying apparatus 200, as also described above. As shown in FIG. 3, three different ophthalmic tools 302, 306, and 310 are connected to the surgical console 102.

The control system 110 includes a processor 316 and a memory 318. The memory 318 may include various types of memory including volatile memory (such as Random Access Memory (RAM)) and non-volatile memory (such as solid state storage). The memory 318 may store machine readable instructions, that when executed by the processor 316, cause the control system 110 to perform various functions. The memory 318 may also include a database signal patterns that are compared with reflected light patterns from an ophthalmic tool to identify a particular ophthalmic tool.

Each of the ophthalmic tools 302, 306, and 310 includes a unique marking 304, 308, and 312, respectively. Specifically, ophthalmic tool 302 includes unique marking 304; ophthalmic tool 306 includes unique marking 308; and ophthalmic tool 310 includes unique marking 312. In the present example, each marking 304, 308, and 312 includes a unique number of rings 311 formed around the circumference of the shaft of the respective ophthalmic tools 302, 306, and 310. As described above, the markings 304, 308, and 312 may represent an optical machine-readable representation of data that represents a unique pattern.

Various mechanisms may be used to make the markings 304, 308, and 312 unique. In the present example, the number of rings 311 for each marking 304, 308, and 312 is varied. In other examples, the distance between rings 311 or the width of the rings 311 may be varied. In some examples, a combination of distance between rings 311, width of rings 311, and number of rings 311 may be used to produce a unique pattern that is detectable by an optical sensor (e.g., optical sensor 220 shown in FIG. 2). In some cases, markings other than rings 311 may be used.

While the markings in the present example are represented as one or more annular rings formed about an exterior surface of the example ophthalmic tools 302, 306, and 310, the scope is not so limited. As explained above, the markings may have other forms. For example, as opposed to forming a complete ring, the marking may extend around only a portion of a shaft of a tool. The marking may be one or more grooves, one or more surface textures, one or more colors, different materials, or a pattern formed on or otherwise arranged on or in a portion of the ophthalmic tool, such as on the shaft of the ophthalmic tool.

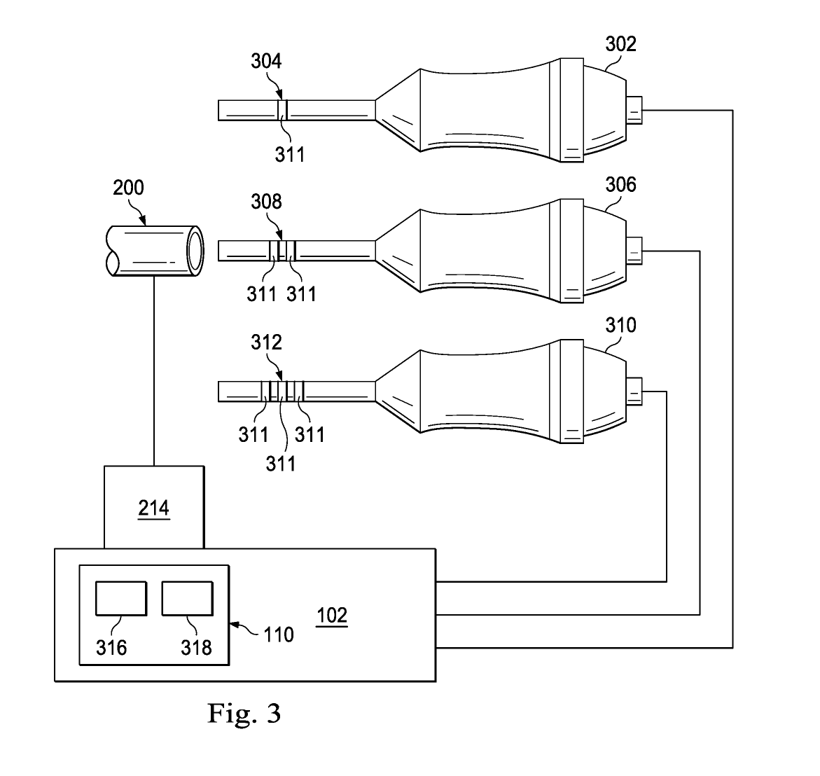

FIGS. 4A and 4B show cross-sectional views of a cannula 401 with a plurality of optical waveguides 402 and 406. The cannula 401 may be similar to the cannula 202 in many respects and therefore some of the same reference numbers are used to denote the similar parts. FIG. 4A illustrates an example in which two optical waveguides 402 and 406 are directed towards the central lumen 206. According to the present example, the cannula 401 includes a first optical waveguide 402 having a first end 404 and a second optical waveguide 406 having a first end 408. Each of the optical waveguide 402 and 406 may have a corresponding light source, beam splitter, and optical sensor in an associated optical transceiver assembly that may be similar to the optical transceiver assembly 214, described above. Having more than one optical waveguide directed towards the central lumen 206 may provide some redundancy to decrease the likelihood that a marking formed on an ophthalmic tool will produce an inaccurate signal and, therefore, reduce the likelihood of an inaccurate identification of the ophthalmic tool. While FIG. 4A shows two optical waveguides 402 and 406, other embodiments may have three optical waveguides or more than three optical waveguides.

FIG. 4B illustrates an example in which a first optical waveguide 402 is directed towards the central lumen 206 and a second optical waveguide 412 has an end 414 that is located at the distal end 203 of the cannula 411. The first optical waveguide 402 is operable to transmit light into the central lumen 206 and receive light therefrom. The second optical waveguide 412 is operable to transmit light to the exterior of cannula 411 adjacent the distal end 203 thereof as well as receive light from the exterior of the cannula 411. As will be described in further detail below, the second optical waveguide 412 may be used to determine whether the cannula 411 is appropriately positioned within the eye.

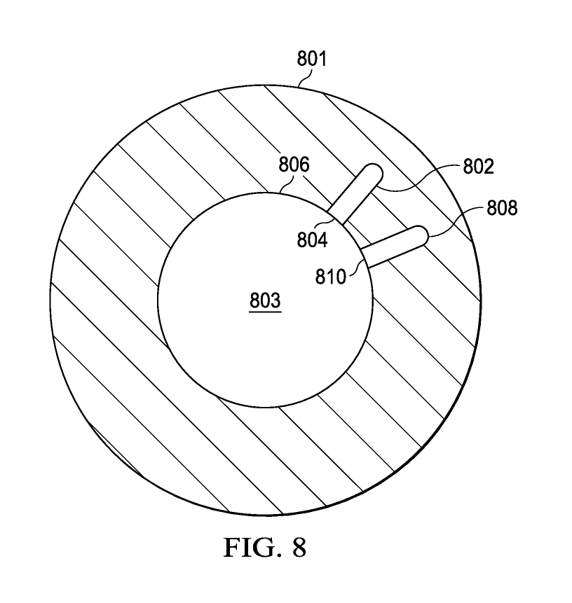

In some implementations, the cannula 401 may include two waveguides that are directed towards the lumen at the same radial plane. That is, in some implementations, a terminal end of two or more optical waveguides may be disposed in a common plane that is transverse to a longitudinal axis of a cannula. FIG. 8 is a transverse cross-sectional view of an example cannula 801, the cross-section being transverse to longitudinal axis 803. Terminal ends 804 and 810 of a first optical waveguide 802 and second optical waveguide 808, respectively, may be angularly offset from each other along a central lumen 806 of the cannula 801 within the common plane. The two optical waveguides 802 and 808 may be positioned close enough to each other so that light being emitted out of one of the optical waveguides is reflected back into both of the waveguides. In such implementations, one of the optical waveguides may be in optical communication with a light source, which may be similar to the light source 216 shown in FIG. 2, and the other optical waveguide may be in optical communication with the optical sensor, which may be similar to optical sensor 220 also shown in FIG. 2. Thus, in some implementations, a beam splitter may not be used. Although an example cannula in which two optical waveguides are disposed is described, more than two waveguides may be disposed within the cannula in order to identify an ophthalmic tool inserted through a central lumen of the cannula.

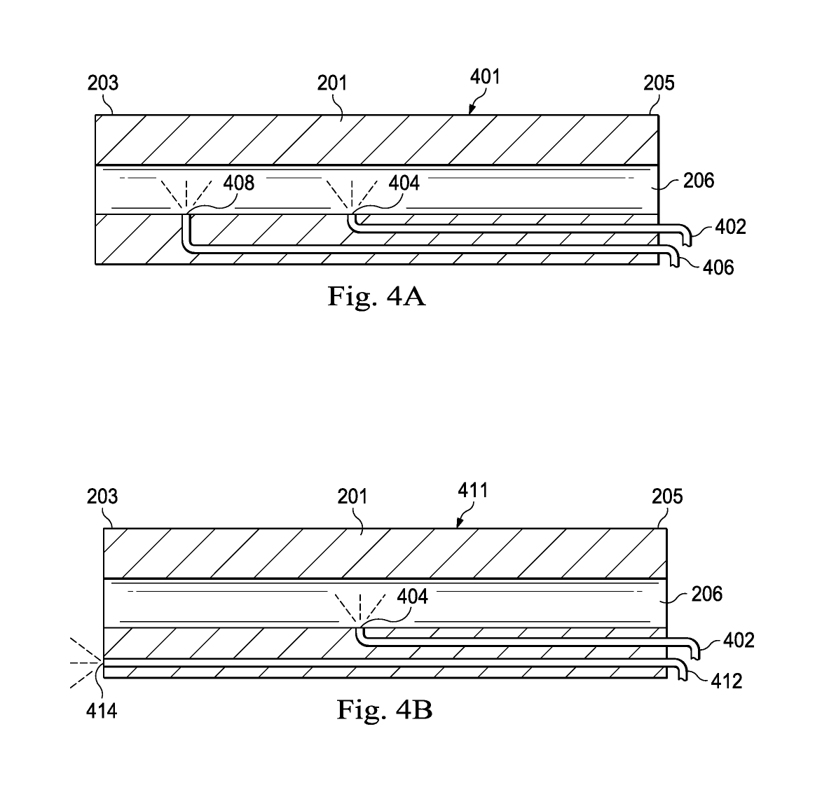

FIGS. 5A and 5B are diagrams showing a cross-sectional view of an optical sensing cannula 511 relative to tissue in an eye 500. FIG. 5A shows the cannula 511 partially within the eye 500, and FIG. 5B shows the cannula 511 fully inserted into the eye 500. As described above, the cannula 511 may be used to determine whether the distal end 503 of a cannula is fully within the interior of the eye 500. FIG. 5A illustrates a case in which the cannula 511 is not fully inserted within the eye 500. Specifically, the distal end 503 of the cannula 511 is positioned within the suprachoroidal space 504 rather than within the vitreous 506 of the eye 500. The choroid 507 is a vascular layer between the sclera 505 and the vitreous 506. The suprachoroidal space 504 is above the choroid 507 between the sclera 505 and the choroid 507. If the distal end 503 of the cannula 511 is within the suprachoroidal space 504, and an infusion tool is inserted into the cannula 511, then the eye may be damaged because fluid is not intended to be injected into the suprachoroidal space 504.

To avoid the situation shown in FIG. 5A and injection of fluid into an inappropriate area of the eye, the cannula 511 includes an optical waveguide 512 similar to those described herein. In the present example, the optical waveguide 512 may direct light to an exterior of the cannula 511 proximate to the distal end 503 thereof. Because the suprachoroidal space 504 is relatively dark and reflects little if any light, the light reflected from the suprachoroidal space and back through the optical waveguide 512 is relatively small. In contrast, when light is directed into the vitreous 506, more light is reflected back through the optical waveguide 512 because the retina and other elements within the vitreous 506 are more reflective. Thus, when the distal end 503 of the cannula 511 is fully within the eye 500, as shown in FIG. 5B, more light is reflected back through the optical waveguide 512.

A control system, which may be similar to the control system 110, may be configured to detect the difference in light levels between light reflected through the optical waveguide 512 when the distal end 503 of the cannula 511 is within the suprachoroidal space 504 and when the distal end 503 of the cannula 511 is within the vitreous 506. For example, if the light level is below a defined first threshold, then the control system may determine that the distal end 503 of the cannula 511 is not within the vitreous 506. Conversely, if the light level is above the first threshold, then the control system 110 may determine that the distal end 503 of the cannula 511 is fully inserted and present within the vitreous 506. Detecting light levels may also be done with an optical waveguide, which may be similar to optical waveguide 208, directed towards a central lumen of the cannula, which may be similar to central lumen 206. The amount of light reflected back through the optical waveguide may be different when the distal end of the cannula is present in the suprachoroidal space 504 as opposed to the vitreous 506. Where an amount of light reflected back through the optical waveguide is below a defined second threshold, a control system such as one or more described herein, may determine that the distal end of the cannula is not disposed in the vitreous. The first threshold may be different than the second threshold.

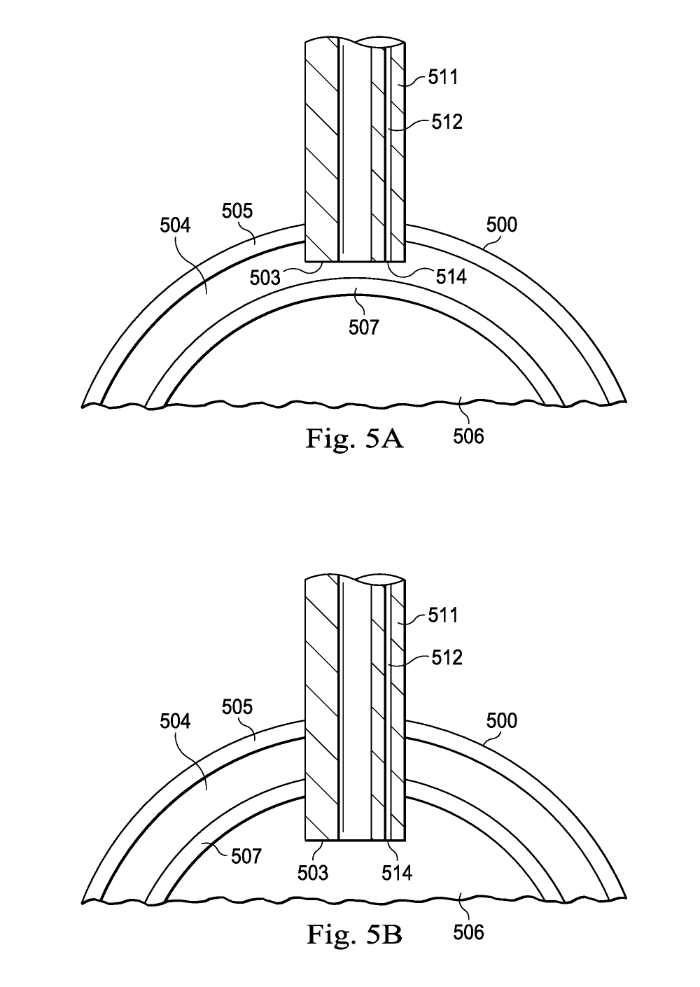

FIG. 6 is a flowchart showing an illustrative method 600 of identifying a tool inserted into an optical sensing cannula. According to the present example, the method 600 includes a step 602 of inserting a cannula into the eye. The cannula may be an optical sensing cannula, such as, for example, the optical sensing cannula 202 described herein. In some implementations, the cannula may be inserted using a trocar as described above.

At step 604, a user inserts an ophthalmic tool into the cannula. The ophthalmic tool may be one of a variety of tools including, for example, without limitation, a vitrectomy probe, a scraper, a forceps, and an aspirator. Other types of ophthalmic tools are contemplated as well. The ophthalmic tools may be connected to a surgical console, such as, for example, surgical console 102. Additionally, the ophthalmic tools may have shafts that have unique markings used to identify the ophthalmic tools.

At step 606, the control system detects a reflected variation of light, such as a reflected light pattern, as the marking on the shaft of the ophthalmic tool passes an end of the optical waveguide that is exposed to a central lumen of the cannula. The optical waveguide may be embedded within the cannula. Specifically, light is directed into the optical waveguide. An optical transceiver assembly, which may be similar to the optical transceiver assembly 214, for example, may include an optical sensor, which may be similar to optical sensor 220, for example. The optical sensor is arranged to detect light that is reflected back through the optical waveguide. The light reflected back through the optical waveguide varies as the marking on the shaft passes the end of the optical waveguide.

The method 600 further includes a step 608 of identifying the ophthalmic tool. The ophthalmic tool maybe identified based on the reflected light pattern detected by the optical sensor. Specifically, the optical sensor may be in communication with a control system, which may be part of a surgical console. The control system may include a database of light patterns associated with different ophthalmic tools. The control system may match the detected light pattern with an entry within the database, thereby identifying the associated ophthalmic tool.

After the ophthalmic tool has been identified, the control system may make certain adjustments to the surgical console. For example, the control system may cause the display of the surgical console to display data related to the ophthalmic tool that is currently within the eye. Additionally, the control system may configure user input devices, such as a foot pedal, so that using such devices operates the ophthalmic tool that is within the eye. Additionally, the control system may disallow certain operations of the ophthalmic tool that are not intended to be used while the ophthalmic tool is in the eye.

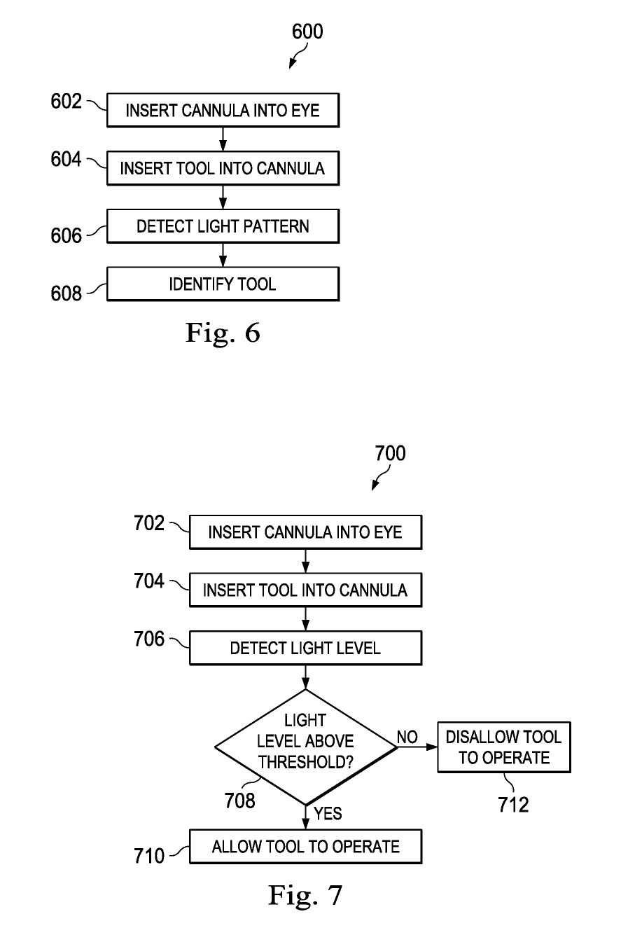

FIG. 7 is an example flowchart showing an illustrative method for using an optical sensing cannula to determine whether the cannula is appropriately positioned. According to the present example, the method 700 includes a step 702 of inserting a cannula into the eye. The cannula may be an optical sensing cannula as described herein. The cannula may be inserted using a trocar as described above.

The method 700 further includes the step 704 of inserting an ophthalmic tool into the cannula. The ophthalmic tool may be one of a variety of tools including a vitrectomy probe, a scraper, a forceps, and an aspirator. Other types of ophthalmic tools are contemplated as well. The ophthalmic tools may be connected to a surgical console. Additionally, the ophthalmic tools may have shafts that have unique markings used to identify the ophthalmic tools.

The method 700 further includes a step 706 of detecting the light level of light reflected back through the optical waveguide within the cannula. Specifically, as described above, an optical transceiver assembly may include a beam splitter that directs light towards an optical sensor. The light sensor may detect a light level of light reflected back through the optical waveguide.

At step 708, the control system determines whether the detected light level is above a defined threshold. If the light level is above the defined threshold, then the control system may allow tools associated with the cannula to operate at step 710. For example, if the cannula is an infusion cannula, or has an infusion tool connected thereto, then the control system may permit the infusion operation. A reflected light level above a defined threshold may be indicative of a proper positioning of the cannula within the eye and, hence, operation of the ophthalmic tool is appropriate.

If, however, the light level is below the defined threshold, then the method 700 proceeds to step 712, at which the control system disallows operation of a tool associated with the cannula. For example, if the cannula is acting as an infusion cannula, then it is desirable to avoid injection a fluid into the eye if the cannula is not properly positioned. Based on the lower light levels, the control system determines that the cannula is not properly positioned. For example, the distal end of the cannula may be within the suprachoroidal space. Thus, the control system may prevent the infusion tool connected to the surgical console from injecting fluid. Additionally, if any other tools are inserted into the cannula, and the cannula is not properly positioned, then the control system may prevent such tools from performing one or more operations while the cannula is not properly positioned.

Although the present disclosure is described in the context of ophthalmology, the scope of the disclosure is not so limited. Rather, the substance of the present disclosure is suitable for many other applications. For example, the present disclosure may be applicable to other types of surgical procedures, such as minimally invasive surgical procedures. Moreover, the scope of the present disclosure is intended to encompass systems and methods for performing tasks with limited access and, particularly, to those involving limited or confined spaces.

Persons of ordinary skill in the art will appreciate that the scope of the present disclosure are not limited to the particular exemplary examples described above. In that regard, although illustrative implementations have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. An apparatus for detecting an ophthalmic tool at a surgical site, the apparatus comprising:
a cannula having an elongated body arranged to be introduced into an eye, the body comprising a lumen therethrough, the lumen arranged to allow a shaft of an ophthalmic tool to fit therethrough;
an optical waveguide having an end facing the lumen;
an optical transceiver assembly in optical communication with the optical waveguide, the optical transceiver assembly comprising:
a light source configured to direct light through the optical waveguide and into the lumen; and
an optical sensor.

2. The apparatus of claim 1, further comprising a control system in communication with the optical sensor, the control system configured to process signals detected by the optical sensor.

3. The apparatus of claim 2, wherein the control system is configured to process a light pattern detected by the optical sensor, the light pattern resulting from a marking on a shaft of the ophthalmic tool passing by the end of the optical waveguide.

4. The apparatus of claim 3, wherein the control system is configured to identify the ophthalmic tool based on the light pattern.

5. The apparatus of claim 4, wherein the control system is configured to configure a surgical console based on the identified ophthalmic tool.

6. The apparatus of claim 2, wherein the control system is configured to determine that an end of the elongated body is not properly positioned at the surgical site based on light detected by the optical sensor.

7. The apparatus of claim 6, wherein the control system is configured to prevent a tool inserted into the lumen from operating in response to determining that the end of the elongated body is not properly positioned.

8. The apparatus of claim 1, wherein the optical waveguide comprises an optical fiber.

9. The apparatus of claim 1, wherein the optical waveguide is embedded within the elongated body.

10. The apparatus of claim 1, further comprising, a plurality of additional optical waveguides each having an end within the lumen.

11. A method for identifying which ophthalmic tool of a plurality of ophthalmic tools is inserted into a cannula, the method comprising:
inserting a cannula into an eye, the cannula comprising an optical waveguide having an end at an interior of the cannula;
inserting an ophthalmic tool into the cannula, the ophthalmic tool comprising a shaft having a marking;
sensing light from within the interior of the cannula through the optical waveguide; and
identifying the ophthalmic tool based on a pattern in the sensed light from within the interior of the cannula, the pattern being produced as the marking passes the endpoint.

12. The method of claim 11, further comprising, in response to identifying the ophthalmic tool, configuring a console for operation with the ophthalmic tool, the ophthalmic tool being connected to the console.

13. The method of claim 11, further comprising:
determining whether the sensed light is above a defined threshold; and
allowing operation of the ophthalmic tool when the sensed light is above the defined threshold.

14. The method of claim 11, further comprising:
determining whether the sensed light is below a defined threshold; and
disallowing operation of the ophthalmic tool when the sensed light is below the defined threshold.

15. The method of claim 11, wherein the marking comprises a one or more rings formed around a circumference of the shaft.

16. The method of claim 15, wherein the marking is made unique based on a variation of at least one of, a number of the rings, a width of at least one ring, and a distance between at least two rings.

17. The method of claim 15, wherein the rings have a different reflectivity than the shaft.

18. A system for detecting an ophthalmic tool at a surgical site, the system comprising:
a console having:
a control system; and
a plurality of ophthalmic tool ports;
a plurality of ophthalmic tools arranged to connect to the ports, each ophthalmic tool comprising a shaft having a unique marking;
a cannula having an optical waveguide with an end directed at an interior of the cannula; and
an optical transceiver assembly in optical communication with the optical waveguide, the optical transceiver assembly comprising:
an optical sensor in communication with the control system;
a light source adapted to direct light into the optical waveguide; and
a beam splitter to direct light from the optical waveguide to the optical sensor.

19. The system of claim 18, wherein the control system comprises a processor and a memory having machine readable instructions that when executed by the processor, cause the control system to:
receive a signal from the optical sensor, the signal being produced in response to the unique marking from one of the plurality of ophthalmic tools passing by the endpoint; and
identifying the one of the plurality of ophthalmic tools based on the signal.

20. The system of claim 19, wherein the control system is configured to configure the console for the one of the ophthalmic tools.
